(19) United States
(12) Patent Application Publication
(10) Pub. No.: US 2023/0233686 A1
(43) Pub. Date: Jul. 27, 2023

(54) CROSSLINKED STARCH DERIVATIVE-BASED MATRIX

(71) Applicant: ROQUETTE FRERES, LESTREM (FR)

(72) Inventors: Julien PARCQ, LILLE (FR); Vincent WIATZ, PREMESQUES (FR); Maxime INGRET, BETHUNE (FR)

(21) Appl. No.: 18/001,496

(22) PCT Filed: Jun. 16, 2021
(86) PCT No.: PCT/EP2021/025211

§ 371 Date: Dec. 12, 2022

(30) Foreign Application Priority Data
Jun. 16, 2020 (EP) 20305659.3

(51) Int. Cl. A61K 47/36; A61K 38/28; A61K 9/00; C02F 1/28; C02F 1/00; B01D 53/02

(52) U.S. Cl. CPC A61K 47/36; A61K 9/0053; A61K 38/28; B01D 53/02; C02F 1/001; C02F 1/285; C02F 2101/308

(57) ABSTRACT

The invention relates to a water insoluble solid crosslinked dextrin-based matrix, wherein the crosslinking agent is sodium trimetaphosphate (STMP), its use and method of preparation.

### FIELD OF THE INVENTION

The present invention is directed to a water insoluble solid crosslinked dextrin-based matrix, wherein the crosslinking agent is sodium trimetaphosphate (STMP), and its use, e.g. for the prolonged release of active ingredients. The invention also relates to a method of preparation of said crosslinked dextrin-based matrix.

### DISCUSSION OF THE PRIOR ART

Hydrogels are well known in the industry especially the pharmaceutical and medical industry. Hydrogels are tridimensional networks of chemically or physically crosslinked hydrophilic polymers. They may be used for different applications including, but not limited to tissue engineering, loading and delivering drugs or siRNA, food thickeners, water treatment.

Wintgens et al. (Carbohydrate Polymers 98 (2013) 896-904; Carbohydrate Polymers 132 (2015) 80-88) report cyclodextrin and cyclodextrin/dextran based hydrogels prepared by crosslinking with sodium trimetaphosphate. According to these articles, the cyclodextrin based hydrogels are promising materials as carriers for bioactive molecules and the cyclodextrin/dextranbased hydrogels are promising as carriers for bioactive molecules and bone regeneration.

International patent application WO 2016/100861 A1 describes crosslinked polysaccharide polymers and their use as flowable hemostatic compositions. The exemplified compositions are based on epichlorhydrin crosslinked maltodextrins. However, the preparation of these compositions is not enabled as the application does not provide any operating procedure enabling the skilled person to work the examples.

Water-insoluble starch derivative-based matrixes for controlled drug release are known in the art. They are generally produced by cross-linking the starch derivative with an organic crosslinking agent. International patent application WO 2019/011964 A1 describes maltodextrins crosslinked with dianhydrides, especially pyromellitic dianhydride and their use in the administration of biological actives, such as insulin. The synthesis of these dianhydride crosslinked maltodextrins is carried out in dimethyl sulfoxide (DMSO) in the presence of triethylamine. The use of organic solvents and hazardous reagents such as trimethylamine is generally to be avoided.

There still remains a need of new materials that are useful as carriers for active ingredients, in particular pharmaceutically active ingredients, and that do not require or at least limit the use of organic solvents and/or hazardous organic reagents.

### SUMMARY OF THE INVENTION

The inventors have found that such material may be provided by reticulating certain dextrins, which will be defined and grouped below under the term “dextrins”, using a special reticulation agent, sodium trimetaphosphate (STMP), the reaction being carried out in an aqueous medium and in the presence of an alkaline agent.

In a first aspect, the present invention therefore relates to a method of preparing a water insoluble crosslinked dextrin-based matrix, comprising the following steps:
a. providing at least one dextrin or at least one dextrin and at least one cyclodextrin,
b. forming the water insoluble crosslinked dextrin-based matrix by crosslinking said dextrin or dextrin and cyclodextrin with sodium trimetaphosphate (STMP) in an aqueous medium containing an alkaline agent, and
c. recovering a mixture of the water insoluble crosslinked dextrin-based matrix and the aqueous medium.

In a second aspect the invention relates to a crosslinked dextrin-based matrix, wherein the dextrin is crosslinked with sodium trimetaphosphate (STMP).

In further aspects, the invention relates to various uses of the crosslinked dextrin-based matrix, e.g. for encapsulating organic compounds, in oral delivery systems and as filter media.

### DETAILED DESCRIPTION OF THE INVENTION

The method of the invention of preparing a water insoluble crosslinked dextrin-based matrix, comprises the following steps:
a. providing at least one dextrin or at least one dextrin and at least one cyclodextrin,
b. forming the water insoluble crosslinked dextrin-based matrix by crosslinking said dextrin or dextrin and cyclodextrin with sodium trimetaphosphate (STMP) in an aqueous medium containing an alkaline agent, and
c. recovering a mixture of the water insoluble crosslinked dextrin-based matrix and the aqueous medium.

The crosslinked dextrin-based matrix obtained according to the invention is water insoluble. Within the meaning of the present invention, the term “water insoluble” means that the matrix may not be dissolved in water at room temperature, i.e. between 18 and 25° C., at pH 7. Preferred crosslinked dextrin-based matrixes according to the invention are insoluble in water at room temperature at a pH ranging from 5 to 9.

The term “dextrin” as used herein includes maltodextrins, glucose syrups having a dextrose equivalent (DE) between 20 and 30, and pyrodextrins. Preferred dextrins within the meaning of the invention are maltodextrins and pyrodextrins. Maltodextrins are obtained by acid and/or enzymatic hydrolysis of starch and have a DE (or Dextrose Equivalent) less than or equal to 20. Pyrodextrins are obtained by dry heating starch under acidic conditions, which generally leads to hydrolysis of the starches followed by reconnection of α-1,6 bonds. These pyrodextrins are referred to white or yellow dextrins, or “British gums”, depending on the temperature, acidity and humidity conditions used. The term “dextrin” as used herein does not include cyclodextrins.

Dextrins suitable to be used in the present invention may be prepared from any type of starch. Non-limiting examples of starch sources include but are not limited to tuber, cereal and legume starches. Non-limiting examples of tuber starches are potato and tapioca starch. Examples of cereal starches include but are not limited to wheat, maize (also called corn) and barley starch. Examples of legume starches include but are not limited to pea, bean, broad bean, horse bean, lentil, lucerne, lupin, and faba bean starch. Therefore, the dextrins used in the invention may be selected from potato, tapioca, wheat, maize, barley, pea, bean, broad bean, horse bean, lentil, Lucerne, lupin, faba bean dextrins and mixtures thereof. Preferably, the dextrins are selected from pea, faba bean and maize dextrins, more preferably from pea and maize dextrins, in particular from pea and maize maltodextrins or pyrodextrins.

In one embodiment, the at least one dextrin is a maize dextrin, in particular a maize pyroextrin.

In another embodiment, the at least one dextrin used in the method of the invention is a leguminous dextrin, preferably this dextrin is derived from a leguminous starch having an amylose content comprised between 25% and 50%, preferably between 30% and 40%, in particular comprised between 35% and 40%, and more preferentially between 35% and 38%, these percentages being expressed as dry weight relative to the dry weight of starch. The leguminous dextrin may be chosen from the group consisting of pea, bean, broad bean, horse bean, lentil, lucerne, lupin, and faba bean dextrins. Preferably, the dextrin is a pea dextrin or a faba bean dextrin, more preferably a pea dextrin.

The term “pea” being here considered in its broadest sense and including in particular: all the wild “smooth pea” varieties and all the mutant “smooth pea” and “wrinkled pea” varieties, irrespective of the uses for which said varieties are generally intended (human consumption, animal nutrition and/or other uses). Said mutant varieties are in particular those known as “r mutants”, “rb mutants”, “rug 3 mutants”, “rug 4 mutants”, “rug 5 mutants” and “lam mutants” as described in the article by C-L HEYDLEY et al. (HEYDLEY C-L (1996) “Developing novel pea starches” Proceedings of the Symposium of the Industrial Biochemistry and Biotechnology Group of the Biochemical Society, pp. 77-87). Preferred pea varieties are smooth pea varieties, especially wild smooth pea varieties.

The at least one dextrin used in the present invention may be selected from maltodextrins, especially leguminous maltodextrins, in particular faba bean or pea maltodextrins, more particularly pea maltodextrins. Preferably, the maltodextrins have a weight average molecular weight chosen within the range of 5 000 to 15 000 Daltons (Da), preferably of 10 000 to 15 000 Da, more preferably 10 000 to 14 000 Da. The weight average molecular weight may be determined by Steric Exclusion Chromatography (SEC).

The use of a maltodextrin, especially a leguminous maltodextrin, in particular a faba bean or pea maltodextrin, more particularly a pea maltodextrin, is particularly interesting because it results in a crosslinked matrix having particular advantageous properties, especially in terms of swelling.

The at least one dextrin used in the invention may also be selected from pyrodextrins, especially maize pyrodextrins.

The at least one dextrin, especially in the case of a pyrodextrin, may be cooked prior to the cross-linking step. The obtained paste may advantageously be cooled to room temperature prior to crosslinking.

The at least one dextrin may be used alone or together with at least one cyclodextrin. The term “cyclodextrin” as used herein includes any of the cyclodextrins known in the art, such as native and unsubstituted cyclodextrins containing 6 to 12 glucose units linked by covalent bonds between carbons 1 and 4, including alpha, beta- and gamma- cyclodextrins containing 6, 7 and 8 glucose units, respectively. Preferred cyclodextrins according to the present invention are alpha-, beta- and gamma-cyclodextrins, native beta-cyclodextrin being the most preferred.

In step b) of the method according to the invention, the at least one dextrin or the at least one dextrin and at least one cyclodextrin are crosslinked with sodium trimetaphosphate (STMP) in an aqueous medium containing an alkaline agent so as to form the water insoluble crosslinked dextrin-based matrix.

Advantageously, step b is carried out in the absence of any organic solvent, i.e. the aqueous medium does not contain any organic solvent. The person skilled in the art can easily determine the reaction conditions and quantities of reagents used.

The term “alkaline agent” as used herein means a basic, ionic salt of an alkali metal or alkaline earth metal, such as hydroxide or a carbonate. The alkaline agent may in particular be chosen from sodium hydroxide, potassium hydroxide, calcium hydroxide, lithium hydroxide, sodium carbonate or mixtures of thereof. A preferred alkaline agent is sodium hydroxide.

Preferably, the alkaline agent is employed in molar ratio of alkaline agent / STMP higher than 1, preferably equal to or higher than 1.5, still preferably equal to or higher than 2.0, still preferably equal to or higher than 2.5. Indeed, the inventors have found that a molar ratio of alkaline agent / STMP, especially NaOH/STMP, below 1 or 2 favors phosphorylation of the dextrins and/or cyclodextrins instead of reticulation. Preferably, this molar ratio is lower than 5.0, still preferably equal to or lower than 4.5, still preferably equal to or lower than 4.0, still preferably equal to or lower than 3.5. It is still preferably equal to about 3, for example equal to 3.1.

Preferably, the alkaline agent is present in an amount so that the pH of the aqueous medium before addition of dextrin and sodium trimetaphosphate is 8 to 14, preferably 10 to 14, in particular about 12.

The crosslinking reaction may be carried out at a temperature between 18° C. and 40° C., preferably between 18° C. and 30° C. Typically, the crosslinking step is carried out at room temperature, i.e. at a temperature between 18 and 25° C.

The reaction time is related to the temperature at which the crosslinking is carried out and may be easily adapted by a person skilled in the art. It is generally between 10 min and 5h, preferably between 15 min and 4 hours.

The STMP / dextrin or STMP / (dextrin and cyclodextrin) ratio may vary depending on the dextrin(s) or dextrin / cyclodextrin mixture used. The selection of a suitable ratio is within the basic expertise of the person skilled in the art. Preferably, this ratio, expressed by dry weight/dry weight, may be equal to or less than 80%, preferably equal to or less than 70%, preferably equal to or less than 60%, preferably between 10% and 60%, and even more preferably between 15% and 50%. It is preferably higher than 15%, still preferably higher than 20%, still preferably equal to or higher than 25%, still preferably higher than 25%, still preferably equal to or higher than 30%, still preferably equal to or higher than 35%, still preferably equal to or higher than 40 still preferably equal to or higher than 45%. It is for example equal to about 50%.

After the water insoluble crosslinked dextrin-based matrix is formed in step c, a mixture of this matrix and the aqueous medium is recovered in step c). This mixture may then be subjected to a step d) in which the water insoluble crosslinked dextrin-based matrix is separated from the aqueous medium. Separation may be carried out by any suitable method known in the art, such as filtration centrifugation, filtration, freeze-drying.

The separated matrix may be dried in a step e). Optionally the separated matrix may be washed, e.g. with demineralized water and/or an alcohol, such as ethanol, after separation step d) and before drying.

In a second aspect, the present invention relates to a water insoluble crosslinked dextrin-based matrix, wherein at least one dextrin or at least one dextrin and at least one cyclodextrin is/are crosslinked with sodium trimetaphosphate. The at least one dextrin and the at least one cyclodextrin are those employed in the method of preparing a crosslinked dextrin-based matrix described above. The dextrin-based matrix according to the invention may be obtained according to this method. Hence, the dextrin-based matrix according to the invention is preferably free of any organic solvent. The expression “free of any organic solvent” within the meaning of the invention means that the matrix does not even contain traces of organic solvent which result from a method of preparation employing one or more organic solvents.

The crosslinked dextrin-based matrix according to the disclosure might contain crosslinked ingredients other than dextrins and cyclodextrins, as long as it does not interfere with the desired properties of said dextrin-based matrix. However, crosslinked dextrin-based matrix according to the disclosure preferably contains no more than 30% dry weigh of crosslinked ingredients other than dextrins and cyclodextrins, preferably no more than 20%, still preferably no more than 10%, still preferably no more than 5%, still preferably no more than 1%, still preferably 0%. As the crosslinked dextrin-based matrix may be obtained according to the method of preparing a crosslinked dextrin-based matrix described above, it advantageously consists of at least one dextrin or at least one dextrin and at least one cyclodextrin which is/are crosslinked with sodium trimetaphosphate. In other words, the dextrin-based matrix according to the disclosure preferably is free of crosslinked ingredients other than dextrins and cyclodextrins.

The crosslinked dextrin-based matrix according to the invention is water insoluble. Within the meaning of the present invention, the term “water insoluble” means that the matrix may not be dissolved in water at room temperature, i.e. between 18 and 25° C., at pH 7. Preferred crosslinked dextrin-based matrixes according to the invention are insoluble in water at room temperature at a pH ranging from 5 to 9.

However, the crosslinked dextrin-based matrix according to the invention swells in water. The swelling capacity of the matrix may be characterized by its swelling index (SI) which is defined as

$$S I \% = \frac{W s - W d}{W d} ∗ 100$$

wherein Wd = dry weight of matrix and Ws = weight of swollen matrix. In order to determine SI%, 1 g of dry matrix is dispersed in 100 mL demineralized water and left 24 h for swelling. After 24 h of contact, the mixture of matrix dispersed in water is centrifuged to separate the supernatant (water) and the bottom layer (swollen matrix or gel). The swollen matrix is then weighed.

The swelling index of the crosslinked dextrin-based matrix according to the invention is preferably at least 200%, more preferably at least 500% and even more preferably at least 600%. It is still preferably equal to or higher than 700%, still preferably equal to or higher than 800%, still preferably equal to or higher than 900%, still preferably equal to or higher than 1000%, still preferably higher than 1000%, still preferably equal to or higher than 1100%, still preferably equal to or higher than 1200%, still preferably equal to or higher than 1300%, still preferably equal to or higher than 1400%, still preferably equal to or higher than 1500%, still preferably equal to or higher than 1600%. It is in general equal to or lower than 4000%, even equal to or lower than 3500%, even equal to or lower than 3000%, even equal to or lower than 2500%, even equal to or lower than 2000%.

Advantageously, the water insoluble cross-linked matrix according to the invention has a negative zeta potential. Preferably, the zeta potential is comprised between -10 mV and -50 mV, more preferably between -20 mV and -30 mV. The zeta potential may be determined by electrophoretic mobility as described in the Examples section.

The water insoluble cross-linked matrix according to the invention may be in the form of particles. The average diameter of the matrix particles may e.g. be comprised between 100 nm and 1000 nm, in particular between 150 nm and 500 nm and more specifically between 200 nm and 300 nm. In order to obtain a suitable particle size, the matrix may be ground. Preferably, the matrix has a polydispersity index of 0.10 to 0.50, preferably 0.15 to 0.45, more preferably 0.20 to 0.40. The average diameter and polydispersity index may be determined by Laser Light Scattering as described in the Examples section.

In one embodiment, the at least one dextrin crosslinked with STMP is a maize dextrin, in particular a maize pyrodextrin.

In another embodiment, the at least one dextrin crosslinked with STMP is a leguminous dextrin, preferably this dextrin is derived from a leguminous starch having an amylose content comprised between 25% and 50%, preferably between 30% and 40%, in particular comprised between 35% and 40%, and more preferentially between 35% and 38%, these percentages being expressed as dry weight relative to the dry weight of starch. The leguminous dextrin may be chosen from the group consisting of pea, bean, broad bean, horse bean, lentil, lucerne, lupin, and faba bean dextrins. Preferably, the dextrin is a pea dextrin or a faba bean dextrin, more preferably a pea dextrin.

The term “pea” being here considered in its broadest sense and including in particular: all the wild “smooth pea” varieties and all the mutant “smooth pea” and “wrinkled pea” varieties, irrespective of the uses for which said varieties are generally intended (human consumption, animal nutrition and/or other uses). Said mutant varieties are in particular those known as “r mutants”, “rb mutants”, “rug 3 mutants”, “rug 4 mutants”, “rug 5 mutants” and “lam mutants” as described in the article by C-L HEYDLEY et al. (HEYDLEY C-L (1996) “Developing novel pea starches” Proceedings of the Symposium of the Industrial Biochemistry and Biotechnology Group of the Biochemical Society, pp. 77-87). Preferred pea varieties are smooth pea varieties, especially wild smooth pea varieties.

In another embodiment, the at least one dextrin crosslinked with STMP is a derived from starch having an amylose content comprised between 25% and 50%, preferably between 30% and 40%, in particular comprised between 35% and 40%, and more preferentially between 35% and 38%, these percentages being expressed as dry weight relative to the dry weight of starch. These dextrins preferably are legume dextrins. These dextrins may be chosen from the group consisting of pea, bean, broad bean, horse bean, lentil, lucerne, lupin, and faba bean dextrins. Preferably, the dextrin is a pea dextrin or a faba bean dextrin, more preferably a pea dextrin.

Water insoluble cross-linked matrixes in which the at least one dextrin is derived from a starch having an amylose content as mentioned above have particular advantageous properties, especially in terms of swelling.

The at least one dextrin crosslinked in the matrix according to invention may be selected from maltodextrins, especially leguminous maltodextrins, in particular faba bean or pea maltodextrins, more particularly pea maltodextrins. Preferably, the maltodextrins have a weight average molecular weight chosen within the range of 5 000 to 15 000 Daltons (Da), preferably of 10 000 to 15 000 Da, more preferably 10 000 to 14 000 Da. The weight average molecular weight may be determined by Steric Exclusion Chromatography (SEC).

Water insoluble cross-linked matrixes in which the at least one dextrin is a maltodextrin, especially a leguminous maltodextrin, in particular a faba bean or pea maltodextrin, more particularly a pea maltodextrin, have particular advantageous properties, especially in terms of swelling.

The at least one dextrin crosslinked in the matrix according to invention may also be selected from pyrodextrins, especially maize pyrodextrins.

The at least one dextrin, especially in the case of a pyrodextrin, may be cooked prior to the cross-linking step. The obtained paste may advantageously be cooled to room temperature prior to crosslinking.

The at least one dextrin may be crosslinked with STMP, either alone or together with at least one cyclodextrin. The term “cyclodextrin” as used herein includes any of the cyclodextrins known in the art, such as native and unsubstituted cyclodextrins containing 6 to 12 glucose units linked by covalent bonds between carbons 1 and 4, including alpha, beta- and gamma-cyclodextrins containing 6, 7 and 8 glucose units, respectively. Preferred cyclodextrins according to the present invention are alpha-, beta- and gamma-cyclodextrins, native beta-cyclodextrin being the most preferred.

The water insoluble crosslinked dextrin-based matrix of the invention may be loaded with active ingredients. Hence, a third aspect of the invention pertains to the use of the water insoluble cross-linked dextrin-based matrix according to the invention as carrier for organic compounds. The matrix according to the invention may indeed loaded with different types of organic compounds, including cationic compounds, nonionic compounds as well as complex compounds such as polypeptides. These organic compounds may in particular be chosen from active ingredients. The term “pharmaceutically active ingredient(s)” within the meaning of the present invention includes small molecule active ingredients as well as large molecule active ingredients. Large molecule active ingredients include without being limited thereto proteins, such as insulin, antibodies, and nucleotides. The active ingredient may e.g. be a pharmaceutically active ingredient, a bioactive ingredient, or a food active ingredients. Preferably, the active ingredient according to the disclosure is a large molecule. Still preferably, the active ingredient according to the disclosure is a protein, still preferably insulin.

The water insoluble cross-linked dextrin-based matrix according to the invention is particularly useful for the sustained release of active ingredients in the human or animal body by oral administration. In a fourth aspect the invention thus relates to an oral delivery system comprising a water insoluble cross-linked dextrin-based matrix according to the invention and an active ingredient, wherein the matrix is loaded with the active ingredient. In other terms, the water insoluble cross-linked dextrin-based matrix according to the invention is used as a carrier for the active ingredient. Suitable active ingredients are those described above.

Because of its ability to retain compounds, the water insoluble cross-linked matrix according to the invention is also useful for capturing pollutants in water or air. It may in particular be used for retaining cationic organic pollutants or metal cations. Cationic organic pollutants include for example cationic small molecule active ingredients and cationic dyes. The crosslinked dextrin-based matrix according to the invention may e.g. be employed as filter media for filtering air or water.

The invention will be better understood in view of the following illustrative and non-limiting examples and Figures.

### FIGURES

FIG. 1 shows the insulin release over time at pH = 1.2 from an insulin loaded matrix according to the invention.

FIG. 2 shows the insulin release over time at pH = 6.8 from an insulin loaded matrix according to the invention.

### EXAMPLES

The following starting materials were used for the synthesis of the cross-linked matrices:
KLEPTOSE Linecaps®17 (Roquette Freres): pea maltodextrin.
Stabilys® A025 (Roquette Freres): maize pyrodextrin.
Stabilys® A053 (Roquette Freres): maize pyrodextrin.
Sodium trimetaphosphate (STMP, Na₃P₃O₉, CAS No. 7785-84-4): Sigma Aldrich, 95% purity.

### Example 1: Synthesis of a Maltodextrin-Based Matrix According to the Invention Crosslinked with 60% STMP

In a glass reactor equipped with a mechanical stirrer, was introduced 105.2 g of Linecaps 17 (residual moisture 4.9 weight%, 100 g dry substance).

20 weight % of NaOH based on dry weight of starch (20 g, 0.5moles) were added under stirring using 10% NaOH solution (200 g).

Reaction was left under stirring at room temperature (~20-25° C.) for 3.5 hours.

60 weight % sodium trimetaphosphate based on dry weight of starch (60 g, 0.196 mole) were added under stirring. The reaction mixture was left for 1.5 h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCI until residual pH reaches 6.5.

The mixture was centrifuged for 15 minutes at 4700 rpm using a VWR Mega Star 1.6 centrifuge. The supernatant was removed and the obtained matrix was washed with demineralized water. After 15 minutes of stirring, the mixture was centrifuged for 15 minutes at 4700 rpm using the same centrifuge as before. The supernatant was removed and the obtained gel was washed with demineralized water 2 more times.

The final matrix was recovered and precipitated in Ethanol under stirring. The obtained white powder was filtered and dried under vacuum. Product was recovered with 59% yield (based on the dry weight of product recovered / the initial amount of dry Linecaps + STMP introduced in the reaction mixture).

### Example 2: Synthesis of a Maltodextrin-Based Matrix According to the Invention Crosslinked with 50% STMP

In a glass reactor equipped with a mechanical stirrer, was introduced 105.2 g of Linecaps 17 (residual moisture 4.9%, 100 g dry substance).

20 weight % of NaOH based on dry weight of starch (20 g, 0.5 moles) were added under stirring using 10% NaOH solution (200 g).

Reaction was left under stirring at room temperature (~20-25° C.) for 3.5 hours.

50 weight % sodium trimetaphosphate based on dry weight of starch (50 g, 0.163 mole) were added under stirring. The reaction mixture was left for 1.5h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCl until residual pH reaches 6.5.

The reaction mixture was centrifuged for 15 minutes at 4700 rpm using a VWR Mega Star 1.6 centrifuge. The supernatant was removed and the obtained gel was washed with demineralized water. After 15 minutes of stirring, the mixture was centrifuged for 15 minutes at 4700 rpm using the same centrifuge as before. The supernatant was removed and the obtained matrix was washed with demineralized water 2 more times.

The final matrix was recovered and precipitated in Ethanol under stirring. The obtained white powder was filtered and dried under vacuum. Product was recovered with 54% yield (based on the dry weight of product recovered / the initial amount of dry Linecaps + STMP introduced in the reaction mixture).

### Example 3: Synthesis of a Maltodextrin-Based Matrix According to the Invention Crosslinked with 40% STMP

In a glass reactor equipped with a mechanical stirrer, was introduced 525,8 g of Linecaps 17 (residual moisture 4.9%, 500 g dry substance).

16 weight % of NaOH based on dry weight of starch (80 g, 2 moles) were added under stirring using 10% NaOH solution (800 g).

Reaction was left under stirring at room temperature (~20-25° C.) for 3.5 hours.

40 weight % sodium trimetaphosphate based on dry weight of starch (200 g, 0.653 mole) were added under stirring. The reaction mixture was left for 1.5h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCl until residual pH reaches 6.5.

The reaction mixture was centrifuged for 15 minutes at 4700 rpm using a VWR Mega Star 1.6 centrifuge. The supernatant was removed and the obtained gel was washed with demineralized water. After 15 minutes of stirring, the mixture was centrifuged for 15 minutes at 4700 rpm using the same centrifuge as before. The supernatant was removed and the obtained matrix was washed with demineralized water 2 more times.

The final matrix was recovered and precipitated in Ethanol under stirring. The obtained white powder was filtered and dried under vacuum. Product was recovered with 59% yield (based on the dry weight of product recovered / the initial amount of dry Linecaps + STMP introduced in the reaction mixture).

### Example 4: Synthesis of a Maltodextrin-Based Matrix According to the Invention Crosslinked with 25% STMP

In a glass reactor equipped with a mechanical stirrer, was introduced 525,8 g of Linecaps 17 (residual moisture 4.9%, 500 g dry substance).

10 weight % of NaOH based on dry weight of starch (50 g, 1,25 moles) were added under stirring using 10% NaOH solution (500 g).

50 g of demineralized water were added to the reaction mixture to allow good stirring conditions.

Reaction was left under stirring at room temperature (~20-25° C.) for 3.5 hours.

25 weight % sodium trimetaphosphate based on dry weight of starch (125 g, 0.408 mole) were added under stirring. The reaction mixture was left for 1.5 h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCl until residual pH reaches 6.5.

The reaction mixture was centrifuged for 15 minutes at 4700 rpm using a VWR Mega Star 1.6 centrifuge. The supernatant was removed and the obtained gel was washed with demineralized water. After 15 minutes of stirring, the mixture was centrifuged for 15 minutes at 4700 rpm using the same centrifuge as before. The supernatant was removed and the obtained matrix was washed with demineralized water 2 more times.

The final matrix was recovered and precipitated in Ethanol under stirring. The obtained white powder was filtered and dried under vacuum. Product was recovered with 58% yield (based on the dry weight of product recovered / the initial amount of dry Linecaps + STMP introduced in the reaction mixture).

### Example 5: Synthesis of a Maltodextrin-Based Matrix According to the Invention Crosslinked with 20% STMP

In a glass reactor equipped with a mechanical stirrer, was introduced 525,8 g of Linecaps 17 (residual moisture 4.9%, 500 g dry substance).

8 weight % of NaOH based on dry weight of starch (40 g, 1 mole) were added under stirring using 10% NaOH solution (400 g).

140 g of demineralized water were added to the reaction mixture to allow good stirring conditions.

Reaction was left under stirring at room temperature (~20-25° C.) for 3.5 hours.

20 weight % sodium trimetaphosphate based on dry weight of starch (100 g, 0.327 mole) were added under stirring. The reaction mixture was left for 1.5 h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCl until residual pH reaches 6.5.

The reaction mixture was centrifuged for 15 minutes at 4700 rpm using a VWR Mega Star 1.6 centrifuge. The supernatant was removed and the obtained gel was washed with demineralized water. After 15 minutes of stirring, the mixture was centrifuged for 15 minutes at 4700 rpm using the same centrifuge as before. The supernatant was removed and the obtained matrix was washed with demineralized water 2 more times.

The final matrix was recovered and precipitated in Ethanol under stirring. The obtained white powder was filtered and dried under vacuum. Product was recovered with 57% yield (based on the dry weight of product recovered / the initial amount of dry Linecaps + STMP introduced in the reaction mixture).

### Example 6: Synthesis of Pyrodextrin-Based Matrices According to the Invention Crosslinked with 60% STMP

Example 6a: In a glass reactor equipped with a mechanical stirrer, was introduced a 20 weight % dry matter slurry composed of 100 g of dry Stabilys® A053 (amount calculated after determination of the residual moisture, 100 g dry substance) and 400 g of demineralized water. This preparation was cooked at 95° C. the slurry became a paste (yellow color). After 30 minutes of cooking at 95° C., the reaction mixture was cooled down to 25° C. prior to addition of sodium hydroxide solution

20 weight % of NaOH based on dry weight of starch (20 g, 0.5 moles) were added under stirring using 10% NaOH solution (200 g).

Reaction was left under stirring at room temperature (~20-25° C.) for 3.5 hours.

60 weight % sodium trimetaphosphate based on dry weight of starch (60 g, 0.196 mole) were added under stirring. The reaction mixture was left for 1.5 h.

After several minutes, jellification of the mixture was observed and stirring was stopped.

After that time, recovery of the crude material was performed.

The solid was crushed and dispersed in a sufficient amount of water to obtain a stirred suspension. Neutralization of the crude was made by addition of HCl until residual pH reaches 6.5.

The reaction mixture was centrifuged for 15 minutes at 4700 rpm using a VWR Mega Star 1.6 centrifuge. The supernatant was removed and the obtained matrix was washed with demineralized water. After 15 minutes of stirring, the mixture was centrifuged for 15 minutes at 4700 rpm using the same centrifuge as before. The supernatant was removed and the obtained matrix was washed with demineralized water 2 more times.

The final matrix was recovered and precipitated in ethanol under stirring. The obtained white powder was filtered and dried under vacuum. Product was recovered with 58% yield (based on the dry weight of product recovered / the initial amount of dry Stabilys® A053 + STMP introduced in the reaction mixture).

Example 6b: Example 3a was repeated by replacing the 20 weight % dry matter slurry of Stabilys® A053 with a 15 weight % dry matter slurry of Stabilys® A025 (75 g of Stabilys® A025 in 425 g of demineralized water). The product was recovered with 68% yield (based on the dry weight of product recovered / the initial amount of dry Stabilys® A025 + STMP introduced in the reaction mixture).

### Example 7: Solubility of Matrices According to the Invention

Solubility of the matrices of Examples 1 to 3 in water was determined according to the following protocol:

250 mg of each matrix was taken in a vial. To each vial, 5 mL of deionized water were added. All samples were stirred periodically and kept under constant observation.

The swelling and dissolution of each sample was observed for up to 72 hours. For checking solubility, the viscosity and transparency of the supernatant liquid (water) was carefully observed through a magnifying glass. The swelling of samples and dissolution can be clearly differentiated by this visual evaluation.

For each matrix, the test was carried out at pH 7, pH 5 (addition of HCl) and pH 9 (addition of NaOH).

All of the samples were insoluble under the test conditions. However, they showed important swelling.

### Example 8: Swelling Capacity of Matrices According to the Invention

The Swelling Index (SI) of the matrices of Examples 2 to 4 was determined according to the following protocol:

1 g (dry weight) of product was dispersed in 100 ml of demineralized water in a graduated cylinder, and left 24 h for swelling. After 24 h of contact the mixture of gel dispersed water was centrifuged to separate the supernatant and the bottom layer (swollen gel). The swollen gel was weighed to determine the amount of water absorbed.

The SI was calculated as described above.

The results are presented in table 1 below.

TABLE 1

| Matrix | SI % |
|---|---|
| Ex. 2 | 1640 |
| Ex. 3 | 1530 |
| Ex. 4 | 1080 |

### Example 9: pH Value, Average Diameter and Polydispersity of Matrices According to the Invention

The pH value of the matrices of Examples 2, 3, 4, and 5 was determined using a pH meter (Orion model 420A).

The average diameter and polydispersity index of the matrices of Examples 2, 3, 4, and 5 were determined by Laser Light Scattering using a 90plus Instrument (Brookhaven, NY, USA) and zeta potential was determined by electrophoretic mobility using the same instrument.

The analyses were carried out on matrix suspensions that were prepared as follows:

1. Preparation of a suspension starting from coarse powder in distilled water at the concentration of 10 mg/ml under stirring at room temperature.

2. Dispersion of the suspension using a high shear homogenizer (Ultraturrax®, IKA, Konigswinter, Germany) for 10 minutes at 24000 rpm.

3. Use of high pressure homogenization for 90 minutes at a back-pressure of 500 bar, using an EmulsiFlex C5 instrument (Avastin, USA) for further size reduction.

4. Purification of homogenized nanosuspension by dialysis (Spectrapore, cellulose membrane, cutoff 12000 Da) to remove synthesis residues potentially present.

5. Storage of the nanosuspensions at 4° C.

The results are presented in table 2 below.

| Matrix | pH | Average diameter (nm) | Polydispersity Index (PI) | Zeta potential (mV) |
|---|---|---|---|---|
| Example 2 | 8.24 | 246.7 | 0.171 | 38.34±2.93 |
| Example 3 | 6.58 | 259.2 | 0.224 | 21.39±1.25 |
| Example 4 | 6.44 | 301.7 | 0.224 | -30.27±1.29 |
| Example 5 | 6.50 | 224.2 | 0.377 | -18.25±1.5 |

### Example 10: Methylene Blue Loading Capacity of Matrices According to the Invention

Methylene blue was used as a model for an organic cationic compound in order to demonstrate the ability of the matrixes of the invention to retain organic cationic compounds.

2 g (dry weight) of crosslinked matrix was dispersed in 100 ml of 10⁻⁵ M methylene blue solution in water and left 24 h for swelling. After 24 h of contact the mixture of gel dispersed in aqueous methylene blue solution was centrifuged to separate the supernatant and the bottom layer (blue colored swollen gel).

Determination of the concentration of residual methylene blue in the supernatant was determined using UV-visible spectrometry.

Methylene blue absorption capacity (in %) was calculated as the ratio of the quantity of methylene blue retained by the matrix / quantity of methylene blue initially introduced * 100. The quantity of methylene blue retained by the matrix corresponds to the difference between the quantity of methylene blue initially introduced and the quantity of methylene blue present in the supernatant.

The results are presented in table 2 below.

TABLE 2

| Matrix | Methylene blue loading capacity % |
|---|---|
| Ex. 1 | 93 |
| Ex. 2 | 88 |
| Ex. 6a (Stabilys® A053) | 84 |
| Ex. 6b (Stabilys® A023) | 78 |

### Example 11: Insulin Loading of Matrices According to the Invention

Insulin from bovine pancreas powder was used to prepare a 2 mg/mL solution in distilled water pH 2.3 adjusted using phosphoric acid. Insulin solution was added to pre-formed aqueous nanosuspensions of the crosslinked matrix (according to the protocol described in example 9) in a weight ratio insulin solution: nanosuspension of 1:5. The mixture was stirred at room temperature for 30 minutes and then centrifuged. The supernatant was separated from the sediment which was collected and freeze-dried.

According to this procedure, freeze-dried insulin-loaded matrices were prepared from the matrices of Examples 1 and 2.

### Insulin Loading Capacity

The loading capacity was determined from the freeze-dried insulin loaded samples according to the following protocol.

2-3 mg of freeze-dried insulin loaded crosslinked matrix was dispersed in 5 mL of distilled water. Sonication (15 minutes, 100 W) and centrifugation treatments were performed so as to allow the release of insulin from the system delivery. Then the supernatant was analyzed for the quantitative determination of insulin.

The quantitative determination of insulin was carried out by High Performance Liquid Chromatography (HPLC) (Perkin Elmer 250B, Waltham, MA) equipped with a spectrophotometer detector (Flexar UV/Vis LC, Perkin Elmer, Waltham, MA). An analytical column C18 (250 mm × 4.6 mm, ODS ultrasphere 5 µm; Beckman Instruments, USA) was used. The mobile phase consisted of a mixture of 0.1 M sodium sulfate in distilled water and acetonitrile (72:28 v/v) filtered through a 0.45 µm nylon membrane and ultrasonically degassed prior to use. Ultraviolet detection was fixed at 214 nm and the flow rate was set to 1 mL/min. The insulin concentration was calculated using external standard method from standard calibration curves. For this purpose, 1 mg of Insulin was weighted, placed in a 10 mL flask, and dissolved distilled water at pH 2.3 adjusted by phosphoric acid to obtain a mother solution. This solution was then diluted using the mobile phase and a series of standard solutions were prepared, consequently injected into the HPLC system. Linear calibration curves were obtained over the concentration range of 0.5-25 µg/mL, linear plotted with regression coefficient of 0.999.

The insulin loading capacity (%) of the delivery systems was calculated as follows:

$$\left[\text{weight of insulin} / \text{weight of freeze dried crosslinked matrix}\right] \times \text{100} \text{.}$$

The results are shown in table 3 below.

TABLE 3

| Matrix | Insulin loading capacity % |
|---|---|
| Ex. 1 | 13.53 ± 0.55 |
| Ex. 2 | 18.10 ± 0.68 |

### Example 12: Insulin Release

### In Vitro Drug Release Kinetics

The in vitro drug release experiment was conducted in a multi-compartment rotating disc (a diffusion cell system comprising a donor chamber separated by a membrane from the donor compartment), constituted from several donor cells on one side separated by a cellulose membrane (Spectrapore, cut-off 50 kDa) from the receiving cells on the other side. The freeze-dried insulin-loaded cross-linked matrix prepared in Example 11 from the matrix of Example 2 was placed in the donor cell (1 mL). The receiving cells were filled by phosphate buffered saline (PBS) solutions at pH 1.2 and pH 6.8 separately. The in vitro release studies were carried out during 24 hours, whereby the receiving phase was withdrawn at regular intervals and replaced with the same amount of new PBS solution. The sampling times investigated were 0.25, 0.5, 0.75, 1, 1.5, 2, 3, 4, 5, 6, 22 and 24 hours. The concentration of insulin in the withdrawn samples was later detected by HPLC.

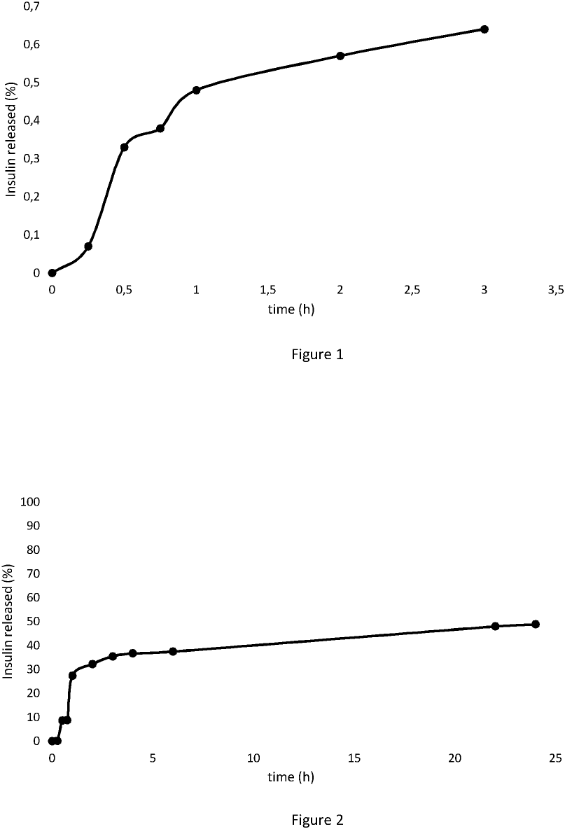

The results are shown in FIGS. 1 and 2. FIG. 1 shows the results up to 3h only as there was no evolution after 3 h.

The cross-linked matrix according to the invention prevents the release of insulin at gastric pH (FIG. 1, pH 1.2), whereas it allows insulin release at intestinal pH (FIG. 2, pH 6.8). In other terms the matrix according to the invention prevents the release of insulin at a pH at which it would be hydrolyzed due to the high acidity of the stomach, and allows the release of insulin in the intestine, where insulin absorption is desired. FIG. 2 also shows that the matrix according to the invention continues to release insulin over a period of several hours.

In addition, the cross-linked matrices according to the invention advantageously allow slow release of insulin. This means that insulin is potentially bioavailable during a longer period of time and that the cross-linked matrices according to the invention are less likely to provoke an abrupt increase of blood insulin after ingestion. The cross-linked matrices according to the invention thus reduce the risk of harmful hypoglycemia caused by abrupt increase of blood insulin.

### In Vivo Experiments

The Freeze-dried insulin-loaded cross-linked matrix prepared in Example 11 from the matrix of Example 2 was administrated to a rat by oral gavage in the stomach. The insulin dose administered was 2.10 mg/kg. Blood samples were collected at different points in time.

The insulin was extracted from plasma samples obtained from the collected blood samples according to the following protocol. To each 100 µl of plasma 100 µl of PBS (pH 7.4), 50 µl of acetonitrile, 20 µl of ethyl paraben, and 3 ml of dichloromethane/n-hexane (1:1 v/v) were added. The mixture was vortexed for 2 min and then centrifuged at 5000 rpm for 10 min. The supernatant was transferred to a test tube. Then 300 µl of 0.05 N HCl were added and the mixture was vortexed for 2 minutes under nitrogen flux. After complete evaporation of the organic phase under nitrogen flux, the remaining supernatant was centrifuged at 15000 rpm for 10 min. A clear supernatant was obtained. The supernatant samples were stored in a freezer at -18° C. and analyzed by HPLC and ELISA.

### HPLC Analysis

HPLC analysis was carried out on a PerkinElmer 250B HPLC system and peaks were integrated using the Chromera software. The experimental HPLC conditions were as follows:
Loop: 20µl
Flow: 1 ml/min
Pressure: 180 bar*
Column: Agilent TC-C18 (2) 5 µm (4.6 mm×150 mm, USA)
λ: 214 nm
Instrument: PerkinElmer 250B, Waltham, MA
Eluent: Mixture of 42 volumes of mobile phase A (solution of 28.4 g of anhydrous sodium sulphate dissolved in 1000 ml of water, pH= 2.3 using phosphoric acid) and 58 volumes of mobile phase B (mixture of 550 ml of mobile phase A and 450 ml of acetonitrile).

The results are presented in table 4.

TABLE 4

| Time (min) | t_{R} (min) | | Area | | Concentration (µg/ml) |
|---|---|---|---|---|---|
| | Bovine Insulin | Internal standard | Bovine Insulin | Internal standard | Bovine Insulin |
| t = 60 min | 11.9 | 6.3 | 796731.4 | 66339.9 | 282.84 |
| t = 180 min | 12.3 | 6.2 | 77600.8 | 59487.6 | 32.13 |

### ELISA Assay

The Elisa assay (Sigma Aldrich ELISA kit) was performed on the samples collected at 15, 60 and 360 min. They are listed in the following table together with the mean absorbance read at 450 nm measured with a Perkin Elmer instrument and the corresponding concentration (µlU/ml). the results are presented in table 5.

TABLE 5

| Sample | Mean absorbance | Concentration* (µlU/ml) | Effective concentration (µlU/ml) |
|---|---|---|---|
| t=360 min | 0.131 | 8.10 | 8.10 |
| t=60 min | 0.127 | 7.358 | 29.42** |
| t=15 min | 0.127 | 7.38 | 7.38 |

*calculated using the insulin calibration curve constructed following the protocol present in the “Certificate of Analysis” of the ELISA kit provided by SIGMA-ALDRICH. The linear regression equation was:
y = 0.0061x + 0.0819 with the correlation coefficient of 0.954.
**The sample was diluted 1:4.

The results of the HPLC analysis and the ELISA assay show that the administered insulin is found in the blood and thus confirm that the matrices according to the invention are useful for oral administration of insulin.

1. A method of preparing a water insoluble crosslinked dextrin-based matrix, comprising the following steps:
a) providing at least one dextrin or at least one dextrin and at least one cyclodextrin,
b) forming the water insoluble crosslinked dextrin-based matrix by crosslinking said dextrin or dextrin and cyclodextrin with sodium trimetaphosphate (ST MP) in an aqueous medium containing an alkaline agent, and
c) recovering a mixture of the water insoluble crosslinked dextrin-based matrix and the aqueous medium.

2. The method according to claim 1, wherein the at least one dextrin is a maltodextrin.

3. The method according to claim 1, wherein the at least one dextrin is a pyrodextrin.

4. The method according to claim 1, wherein the crosslinking is carried out in the absence of any organic solvent.

5. A water insoluble crosslinked dextrin-based matrix, wherein at least one dextrin is or at least one dextrin and at least one cyclodextrin are is/are crosslinked with sodium trimetaphosphate.

6. The cross-linked dextrin-based matrix according to claim 5, wherein the at least one dextrin is a maltodextrin.

7. The cross-linked dextrin-based matrix according to claim 5, wherein the at least one dextrin is a pyrodextrin.

8. Use of the cross-linked dextrin-based matrix according to claim 1 as carrier for organic compounds.

9. Use according to claim 8, wherein the organic compounds are selected from active ingredients, in particular chosen from pharmaceutically active ingredients, bioactive ingredients, and food active ingredients.

10. Use according to claim 9, wherein the active ingredient is insulin.

11. An oral delivery system comprising a water insoluble cross-linked dextrinbased matrix according to claim 1 and an active ingredient, wherein the matrix is loaded with the active ingredient.

12. The oral delivery system according to claim 11, wherein the active ingredient is an active ingredient, in particular chosen from pharmaceutically active ingredient, a bioactive ingredient or a food active ingredient active ingredient.

13. The oral delivery system according to claim 12, wherein the active ingredient is insulin.

14. Use of the water insoluble cross-linked dextrin-based matrix according to claim 1 for capturing pollutants in water or air.
